(19) United States
(12) Patent Application Publication
(10) Pub. No.: US 2012/0125338 A1
(43) Pub. Date: May 24, 2012

(54) DUAL TUBE MASK WITH NASAL CANNULA

(76) Inventor: Alireza Yarahmadi, Mason City, IA (US)

(21) Appl. No.: 12/951,728

(22) Filed: Nov. 22, 2010

(51) Int. Cl. A61M 16/06

(52) U.S. Cl. 128/205.25

(57) ABSTRACT

A dual tube mask system having a shell attached to a frame where the shell has a primary port that is connected to a machine for blowing breathable gas to the mask through a tube. A nasal cannula is disposed within the shell and is connected to a source of oxygen.

### BACKGROUND OF THE INVENTION

This invention is directed toward a mask suitable for the delivery of non-invasive positive pressure ventilation and for nasal CPAP treatment of sleep disordered breathing conditions such as obstructive sleep apnea.

Obstructive Sleep Apnea (OSA) is a disease characterized by excessive daytime sleepiness, loud snoring, and daytime irritability. Other effects of OSA can include depression, high blood pressure, serious heart conditions, sexual problems, memory lapses, intellectual deterioration, and morning headaches. The treatment of OSA by the application of nasal Continuous Positive Air Pressure (CPAP) is well known in the art and involves the use of a machine to blow pressurized breathable gas (typically room air) to either the nose or nose of a patient while they sleep.

Non-invasive Positive Pressure Ventilation (NIPPV) is another form of treatment for breathing disorders. In its most basic form, NIPPV involves a relatively higher pressure of gas being provided in the patient's mask during the aspiratory phase of respiration and a relatively lower pressure during the expiratory phase.

Other treatments, particularly for asthma, emphysema, congestive heart failure, and the like include supplying oxygen to the mask in addition to pressurized breathing gas. Typically, an oxygen tube connected to a source of oxygen is connected to a mask and provides oxygen (O₂) through a port to the shell of the mask.

While these methods have assisted in the treatment of various breathing conditions there are still deficiencies that exist. More specifically, the oxygen provided to the mask becomes diluted because some oxygen leaks through the vent/exhaust openings of the mask. The problem can be addressed by increasing the pressure of the breathable gas blown to the mask but there are limits as to the amount of pressure that can be used as at certain pressure levels the mask becomes uncomfortable or unbearable for the patient to wear. Specifically, higher CPAP/BPAP pressure can lead to adverse effects and consequences such as higher mask leakage, increased nasal drying or congestion, pressure sores on the bridge of the nose, difficulty exhaling, and higher machine noise, among others.

Furthermore, in some conditions CPAP/BIPAP (Bilevel Positive Airway Pressure) therapy alone fails to provide satisfactory oxygenation, particularly in patients with severe sleep apnea and the combination of OSA with conditions including COPD (chronic obstructive pulmonary disease), CHF (congestive heart failure), pulmonary fibrosis, neuromuscular disorders, chronic narcotic use, or central hypoventilation syndrome are among many conditions that may require the need for supplemental administration of oxygen during the titration process. Treatment of patients experiencing such conditions often is provided by introducing oxygen into the CPAP/BIPAP mask or hose, which in current clinical practice is achieved by adding oxygen into a small hole of the CPAP/BIPAP mask or introducing oxygen via the CPAP/BIPAP hose. However, such treatment according to current practice is compromised because the added oxygen flow to the CPAP/BIPAP hose or mask becomes diluted by the CPAP/BIPAP flow and is also subject to leak, and thus patients rarely provided with the benefits of the supplemental oxygen. Additionally, adding oxygen to the patient's mask according to current practice is impractical and ineffective as the connection between the source of oxygen and the mask is susceptible to disconnection, as the patient can easily disconnect the oxygen from the mask as the patient changes positions during sleep; and such incidents are commonly the subject of patient complaints in CPAP/BIPAP systems providing an oxygen line connected to the mask. Finally, existing oxygen and CPAP/BIPAP systems are characterized by additional disadvantages attendant to their requirements of two separate tubes on the bed and two separate machines at the bedside in terms of space, convenience, and oxygen disconnection. Accordingly, there exists a need in the art for a mask that addresses these deficiencies.

An objective of the present invention is to provide a mask system that provides a desired amount of oxygen to a patient at a low pressure.

Another objective of the present invention is to provide a mask system that has fewer exposed tubes and is more convenient for use and decreases the chance of oxygen line disconnection by patient movements.

Yet another objective of the present invention is to provide a mask system that provides a higher oxygen saturation during inhalation with the help of CPAP/BIPAP pressure.

A further objective of the present invention is to provide a mask system which delivers supplemental oxygen directly to a patient's nose in order to minimize dilution and leakage to provide higher oxygenation with minimum CPAP/BIPAP pressure.

A further objective of the present invention is to provide a mask system which delivers supplemental oxygen directly to a patient's nose that is adjustable.

Yet another objective of the present invention is to provide a mask system which reduces pressure related side effects and provides higher patient compliance rates.

These and other objectives will be apparent to one of ordinary skill in the art based upon the written description, drawings, and claims.

### BRIEF SUMMARY OF THE INVENTION

A dual tube mask system having a frame with a shell attached to the frame. The shell has a primary port that is connected to a machine for blowing breathable gas by a tube. A nasal cannula dwells within the shell and is connected to a source of oxygen by an oxygen tube.

### BRIEF DESCRIPTION OF THE DRAWINGS

FIG. 1 is a side view of a mask system;

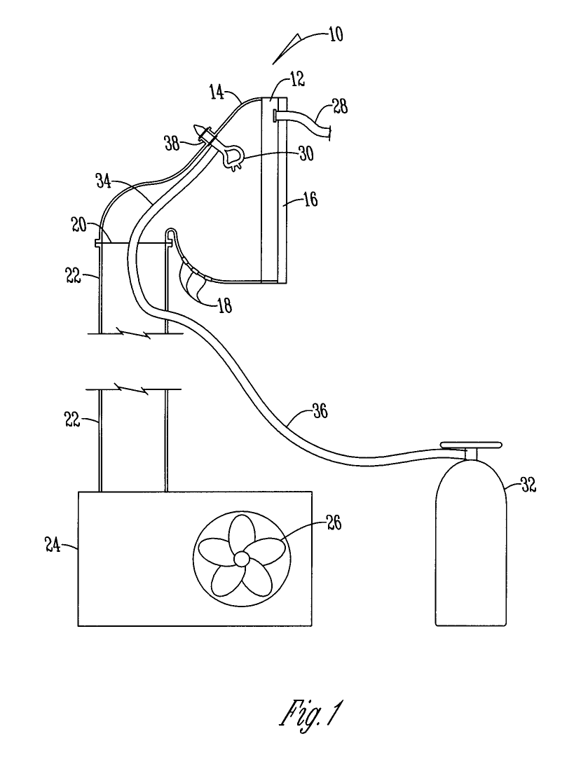

FIG. 2 is a side view of an alternative mask system;

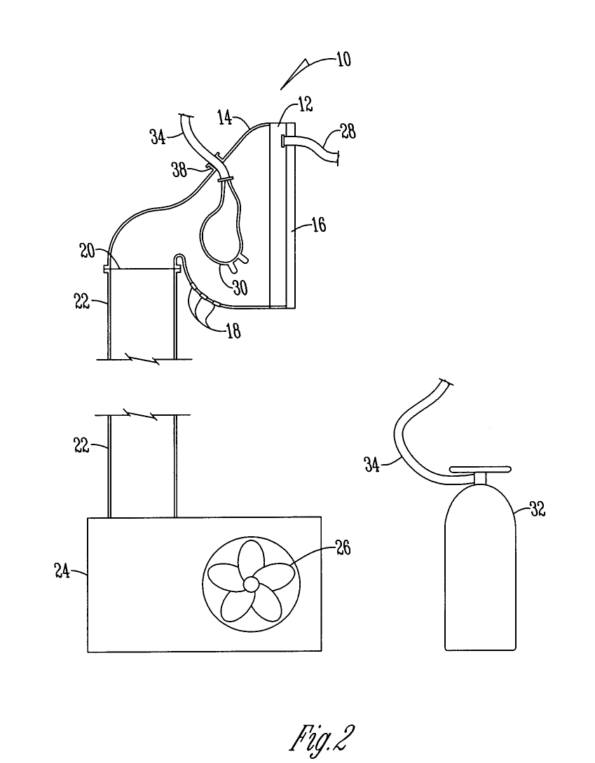

FIG. 3 is a side view of an alternative mask system; and

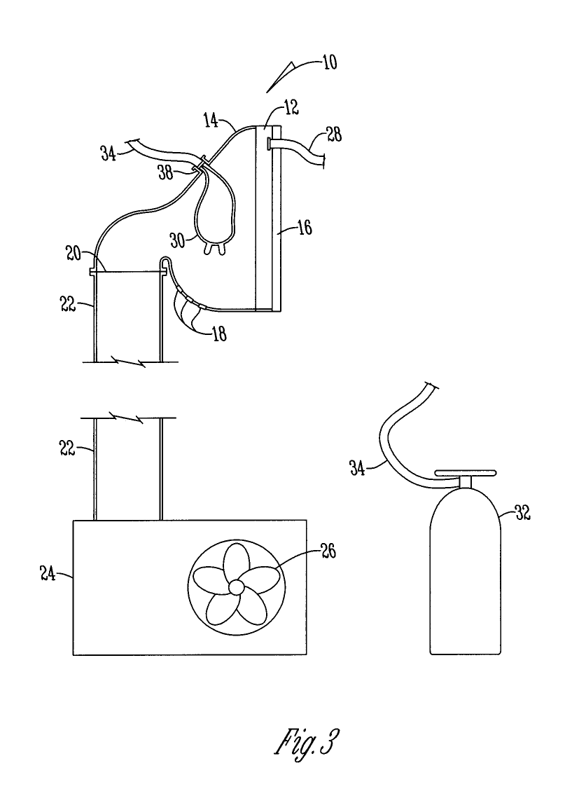

FIG. 4 is a side view of an alternative mask system.

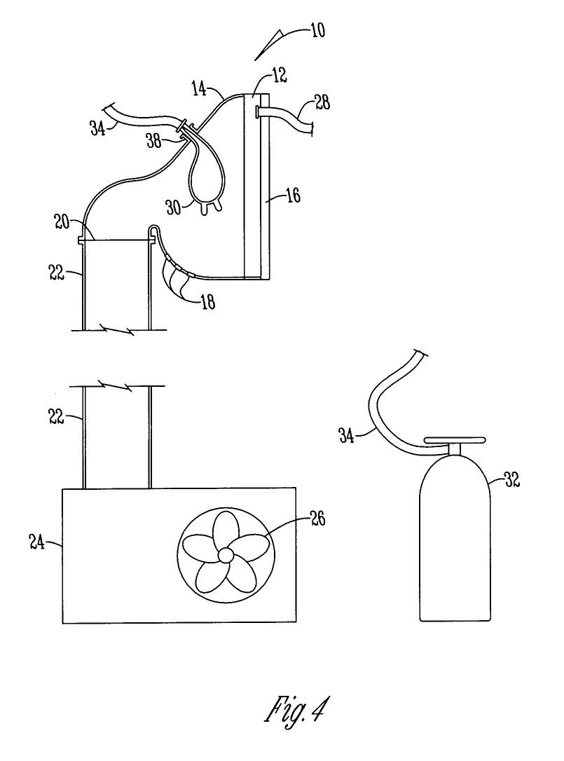

### DETAILED DESCRIPTION OF THE PREFERRED EMBODIMENT

Referring to the Figures, the dual tube mask system 10 includes a frame 12 with a shell 14 attached to one surface of the frame 12 and preferably a cushion 16 attached to the opposite surface of the frame 12. The shell 14 has at least one vent 18 to permit the exhaust of CO2 from the mask 10 as the patient exhales. A primary port 20 is positioned on the shell 14 wherein the primary port 20 is formed to connect with a tube 22 that provides pressurized breathable gas (i.e., room air) from a machine 24 with a blower 26. At least one strap 28 is removably attached to the frame 12 and/or shell 14 at one end in any conventional manner and is preferably fixedly attached to the frame 12 and/or shell 14 at the opposite end.

In one embodiment a nasal cannula 30 dwells within the shell 14 and is connected to a source of oxygen 32 through an oxygen tube 34. The oxygen tube 34 extends from the cannula 30 through the primary port 20 and into tube 22. The oxygen tube 34 extends within tube 22 and is connected to the source of oxygen 32 either through machine 24 or a slit or opening 36 in tube 22. In this embodiment there are fewer tubes exposed and thus the mask system is more convenient for a patient's use.

In another embodiment the cannula 30 dwells within the shell 14 and is connected to an oxygen tube 34. The cannula 30 is connected to the oxygen tube 34 through the use of a secondary port 38 in the shell 14 of the mask. For example, the cannula 30 is connected to an inner end of the secondary port 38 while the oxygen tube 34 is connected to the outer end of the secondary port 38. Alternatively the cannula 30 is directly connected to the oxygen tube 34 wherein either the cannula 30 or the oxygen tube 34 extend through the secondary port 38 such that the oxygen tube 34 is connected to the source of oxygen 32 such that the length of the nasal cannula 30 can be adjusted from the outside of the mask or shell 14 by the patient.

In operation, the cannula 30 is connected to the oxygen tube 34 and to a patient's nasal cavity. Then the mask 10 is placed over the patient's nose of the patient's nose and mouth and secured to the patient's head by strap 28. Once the mask 10 and cannula 30 are in place the machine 24 is activated to blow pressurized breathable gas from the machine 24 through tube 22 to the mask 10. The source of oxygen 32 is also activated to provide oxygen directly to the patient through the oxygen tube 34 and the cannula 30.

The following representative clinical case presentations illustrate the effectiveness of the dual tube system of the present invention providing the delivery of oxygen (O₂) with the cannula 30 via the oxygen tube 34 within the shell 14 of the mask system 10 in concert with the administration of breathable gas from the machine 24 through tube 22 to the mask 10:

### Case Presentations:

### Case 1

A 61 year-old female with complaints of excessive fatigue, history of loud snoring, and congestive heart failure. Patient is on Lortab for shoulder pain. AHI was 53.5 in first sleep study. During the second night of polysomnogram for CPAP titration, Patient failed CPAP due to continuous desaturation and frequent apneas and hypopeneas. Thereafter, Patient switched to BIPAP. Despite increasing BIPAP pressure to 22/18, Patient continued to have desaturation until 3 lit of O2 added to her BIPAP. O2 saturation stayed in low 90's and high 80's on this setting but patient was nervous and unable to tolerate this pressure especially due to leakage from her full-face mask.

Interval Statistics
Treatment Parameters and Results

| Treatment | TIB | TST | Slp Eff | Apneas | | Hypopneas | | AHI | RDI | Ar + Aw | | Min |
|---|---|---|---|---|---|---|---|---|---|---|---|---|
| RxI/RxE/RxO2 | Minutes | Minutes | Percent | Count | Index | Count | Index | Index | Index | Count | Index | SpO2 |
| 8/4/0 | 22 | 6 | 27.3 | 1 | 10 | 0 | 0 | 10 | 10 | 3 | 30 | 83 |
| 9/5/0 | 22 | 14.5 | 65.9 | 3 | 12.4 | 7 | 29. | 41.4 | 41.4 | 12 | 49.7 | 86 |
| 10/6/0 | 17 | 13 | 76.5 | 4 | 3.2 | 9 | 41.5 | 60 | 60 | 11 | 50.8 | 88 |
| 11/7/0 | 44 | 38 | 86.4 | 2 | 3.2 | 1 | 1.6 | 4.7 | 4.7 | 2 | 3.2 | 86 |
| 12/8/0 | 28 | 15.5 | 55.4 | 0 | 0 | 0 | 0 | 0 | 0 | 12 | 46.5 | 87 |
| 14/10/0 | 52.5 | 27 | 51.4 | 8 | 17.8 | 1 | 2.2 | 20 | 20 | 24 | 53.3 | 84 |
| 16/12/0 | 47 | 43.5 | 92.6 | 0 | 0 | 0 | 0 | 0 | 0 | 4 | 5.5 | 89 |
| 13/9/0 | 26.5 | 1 | 3.8 | 0 | 0 | 0 | 0 | 0 | 0 | 1 | 60 | 89 |
| 18/14/0 | 14 | 14 | 100 | 5 | 21.4 | 1 | 4.3 | 25.7 | 25.7 | 0 | 0 | 90 |
| 19/15/0 | 28.5 | 28 | 98.2 | 4 | 8.6 | 0 | 0 | 8.6 | 8.6 | 1 | 2.1 | 93 |
| 20/16/0 | 25 | 19.5 | 78 | 2 | 6.2 | 0 | 0 | 6.2 | 6.2 | 6 | 18.5 | 92 |
| 21/17/0 | 60.5 | 56 | 92.6 | 3 | 3.2 | 1 | 1.1 | 4.3 | 4.3 | 9 | 9.6 | 90 |
| 23/19/0 | 6 | 3 | 50 | 0 | 0 | 0 | 0 | 0 | 0 | 2 | 40 | 92 |
| 22/18/0 | 32 | 30.5 | 95.3 | 0 | 0 | 0 | 0 | 0 | 0 | 2 | 3.9 | 91 |
| — | — | — | — | — | — | — | — | — | — | — | — | — |
| — | — | — | — | — | — | — | — | — | — | — | — | — |

Patient was scheduled for a second night of CPAP/BIPAP titration. Titration started with Dual mask and 2 lit of O2. With CPAP of 10 Cm AHI was 1.2 with good patient's tolerance and O2 saturation.

Interval Statistics
Treatment Parameters and Results/with Dual Mask

| Treatment | TIB | TST | Sleep Efficiency | Apneas | | Hypopneas | | AHI | RDI | Ar + Aw | | Min |
|---|---|---|---|---|---|---|---|---|---|---|---|---|
| RxI/RxE/RxO2 | Minutes | Minutes | Percent | Count | Index | Count | Index | Index | Index | Count | Index | SaO2 |
| 6/6/0 | 98 | 86 | 87.8 | 2 | 1.4 | 1 | .7 | 2.1 | 2.1 | 7 | 4.9 | 92 |
| 8/8/0 | 178.5 | 150.5 | 84.3 | 0 | 0 | 3 | 1.2 | 1.2 | 1.2 | 52 | 20.7 | 94 |
| 10/10/0 | 127 | 98.5 | 77.6 | 0 | 0 | 2 | 1.2 | 1.2 | 1.2 | 10 | 6.1 | 95 |
| 0/0/0 | .5 | 0 | 0 | 0 | 0 | 0 | 0 | 0 | 0 | 0 | 0 | 0 |
| — | — | — | — | — | — | — | — | — | — | — | — | — |
| — | — | — | — | — | — | — | — | — | — | — | — | |

### Case 2

A 51-year old male with history of COPD and daytime fatigue. Patient underwent a split night PSG. Patient's AHI was 35 with minimum desaturation in low 60's. CPAP was applied. Right away patient started to have central apneas, therefore BIPAP was applied. Patient is a significant mouth breather, so technician tried every full-face mask with or without chinstrap. None of them kept a good seal due to patient's beard. Low tidal volume and events still noted due to mouth breathing. At BIPAP pressure of 15/11, 2 lit of O2 was applied but it did not improve the saturation. Technician was unable to establish a final pressure due to numerous central apneas and desaturation.

Interval Statistics
Treatment Parameters and Results

| Treatment | TIB | TST | Slp Eff | Apneas | | Hypopneas | | AHI | RDI | Ar + Aw | | Min |
|---|---|---|---|---|---|---|---|---|---|---|---|---|
| RxI/RxE/RxO2 | Minutes | Minutes | Percent | Count | Index | Count | Index | Index | Index | Count | Index | SpO2 |
| 4/0/0 | 12 | 4.5 | 37.5 | 0 | 0 | 4 | 53.3 | 53.3 | 53.3 | 1 | 13.3 | 86 |
| 6/0/0 | 35.5 | 13.5 | 38. | 13 | 57.8 | 7 | 31.1 | 88.9 | 88.9 | 4 | 17.8 | 80 |
| 8/0/0 | 10 | 10 | 100 | 12 | 73.8 | 1 | 6 | 78 | 78 | 7 | 42 | 79 |
| 10/0/0 | 7 | 6.5 | 92.9 | 8 | 73.8 | 0 | 0 | 73.8 | 73.8 | 7 | 64.6 | 78 |
| 10/6/0 | 8.5 | 8.5 | 100 | 10 | 70.6 | 0 | 0 | 70.6 | 70.6 | 10 | 70.6 | 75 |
| 12/8/0 | 17 | 17 | 100 | 20 | 70.6 | 4 | 14.1 | 84.7 | 84.7 | 4 | 14.1 | 74 |
| 14/10/0 | 16 | 15.5 | 96.9 | 8 | 31. | 8 | 31. | 61.9 | 61.9 | 0 | 0 | 82 |
| 15/11/0 | 20 | 19 | 95 | 17 | 53.7 | 2 | 6.3 | 60 | 60 | 8 | 25.3 | 80 |
| — | — | — | — | — | — | — | — | — | — | — | — | — |

Around 0300, Dual mask with 2 Liter of O2 was applied and titration started with BIPAP of 8/4. At BIPAP pressure of 12/8, SPO2 remained in low 90's with AHI of 1.4.

### Case 3

A 58-year old male with congestive heart failure, excessive daytime somnolence and witnessed apnea. Patient's previous polysomnogram was suggestive for mixed obstructive and central apneas with AHI of 44.5. Patient failed CPAP titration in first sleep study due to recurrent arousal and desaturation. Patient had 4 pillows and a rolled up towel underneath his head. BIPAP was applied at the start of the second study at an initial pressure of 8/4 cm H2O. Patient initially chose a medium size full-face mask, but switched to a size large later on in the study due to mask leakage issues. Patient had difficulty tolerating BIPAP pressure of 20/16 cm H2O.

Interval Statistics
Treatment Parameters and Results

| Treatment | TIB | TST | Slp Eff | Apneas | | Hypopneas | | AHI | RDI | Ar + Aw | | Min |
|---|---|---|---|---|---|---|---|---|---|---|---|---|
| RxI/RxE/RxO2 | Minutes | Minutes | Percent | Count | Index | Count | Index | Index | Index | Count | Index | SpO |
| 8/4/0 | 15.5 | 15.5 | 100 | 1 | 3.9 | 4 | 15.5 | 19.4 | 19.4 | 0 | 0 | 81 |
| 10/6/0 | 17 | 17 | 100 | 1 | 13.3 | 4 | 14.1 | 17.6 | 17.6 | 0 | 0 | 76 |
| 12/8/0 | 11 | 11 | 100 | 1 | 5.5 | 8 | 43.6 | 49.1 | 49.1 | 4 | 21.8 | 78 |
| 14/10/0 | 31 | 31 | 100 | 1 | 1.9 | 4 | 7.7 | 9.7 | 9.7 | 0 | 0 | 79 |
| 16/12/0 | 24.5 | 24 | 98. | 1 | 2.5 | 3 | 7.5 | 10 | 10 | 2 | 5 | 79 |
| 18/14/0 | 14.5 | 14.5 | 100 | 3 | 12.4 | 5 | 20.7 | 33.1 | 33.1 | 5 | 20.7 | 70 |
| 20/16/0 | 23.5 | 4.5 | 19.1 | 1 | 13.3 | 1 | 13.3 | 26.7 | 26.7 | 3 | 40 | 71 |

A Dual mask was applied and titration started at pressure of 6/4 with 2 lit of O2. At the pressure of 12/8 with 3 lit of O2, Biflex of 3 and a humidity rate of 2 most of abnormal respiratory events resolved. This pressure was easily tolerable, without any significant leakage.

Interval Statistics
Treatment Parameters and Results/with Dual Mask

| Treatment | TIB | TST | Slp Eff | Apneas | | Hypopneas | | AHI | RDI | Ar + Aw | | Min |
|---|---|---|---|---|---|---|---|---|---|---|---|---|
| RxI/RxE/RxO2 | Minutes | Minutes | Percent | Count | Index | Count | Index | Index | Index | Count | Index | SpO2 |
| 6/4/0 | 51 | 3 | 5.9 | 0 | 0 | 5 | 100 | 100 | 100 | 1 | 20 | 89 |
| 8/6/0 | 117.5 | 83.5 | 71.1 | 2 | 1.4 | 9 | 6.5 | 7.9 | 7.9 | 34 | 24.4 | 87 |
| 10/8/0 | 21.5 | 19.5 | 90.7 | 0 | 0 | 0 | 0 | 0 | 0 | 10 | 30.8 | 92 |
| 12/8/0 | 96 | 86.5 | 90.1 | 0 | 0 | 2 | 1.4 | 1.4 | 1.4 | 9 | 6.2 | 92 |
| — | — | — | — | — | — | — | — | — | — | — | — | — |

### Case 4:

A 68-year old female with history of witnessed apneas and CHF. Patient is a chronic smoker. Patient scheduled for a split night study and her AHI was 38 during the first part of sleep study with desaturations in low 70's. CPAP was applied around 2320. Pressure of 7 cm H2O was satisfactory until patient aroused, and then patient started to have many centrals, therefore BIPAP was applied. At the BIPAP pressure of 14/10, 2 lit of O2 was added and it was increased to 3 lit at BIPAP pressure of 18/12. BIPAP and CPAP both failed and technician was unable to establish a final pressure due to numerous centrals, mainly in REM. Patient requested to end the study around 0200 because the mask was hurting her face. Sleep technician tried different type of masks and a small Quattro full face was used because patient felt most comfortable. Patient was a dominant mouth breather. Patient slept in the supine position for entire study.

Interval Statistics
Treatment Parameters and Results

| Treatment | TIB | TST | Slp Eff | Apneas | | Hypopneas | | AHI | RDI | Ar + Aw | | Min |
|---|---|---|---|---|---|---|---|---|---|---|---|---|
| RxI/RxE/RxO2 | Minutes | Minutes | Percent | Count | Index | Count | Index | Index | Index | Count | Index | SpO2 |
| 4/0/0 | 12.5 | 8.5 | 68 | 4 | 28.2 | 7 | 49.4 | 77.6 | 77.6 | 4 | 28.2 | 88 |
| 6/0/0 | 15.5 | 15.5 | 100 | 4 | 15.5 | 2 | 7.7 | 23.2 | 23.2 | 2 | 7.7 | 87 |
| 7/0/0 | 23 | 23 | 100 | 1 | 20 | 0 | 0 | 2.6 | 2.6 | 0 | 0 | 88 |
| 8/0/0 | 12 | 6 | 50 | 2 | 20 | 2 | 20 | 40 | 40 | 0 | 0 | 88 |
| 9/0/0 | 9 | 9 | 100 | 13 | 86.7 | 1 | 6.7 | 93.3 | 93.3 | 7 | 46.7 | 83 |
| 10/6/0 | 18 | 18 | 100 | 23 | 76.7 | 3 | 10 | 86.7 | 86.7 | 22 | 73.3 | 81 |
| 12/8/0 | 15 | 14 | 93.3 | 22 | 94.3 | 1 | 4.3 | 98.6 | 98.6 | 12 | 51.4 | 80 |
| 14/10/0 | 8.5 | 8.5 | 100 | 11 | 77.6 | 3 | 21.2 | 98.8 | 98.8 | 3 | 21.2 | 85 |
| 16/12/0 | 14.5 | 14.5 | 100 | 19 | 78.6 | 1 | 4.1 | 82.8 | 82.8 | 4 | 16.6 | 80 |
| 18/12/0 | 33 | 26 | 78.8 | 19 | 43.8 | 4 | 9.2 | 53.1 | 53.1 | 5 | 11.5 | 86 |
| — | — | — | — | — | — | — | — | — | — | — | — | — |
| — | — | — | — | — | — | — | — | — | — | — | — | — |
| — | — | — | — | — | — | — | — | — | — | — | — | — |
| — | — | — | — | — | — | — | — | — | — | — | — | — |

In Patient's second night of polysomnogran, she started on BIPAP titration with Dual mask and 2 lit of O2. At the pressure of 14/10 with 3 lit of O2 most apneas, hypopneas and snoring resolved.

Sequence Statistics
Treatment Parameters and Results/with Dual mask

| Treatment | TIB | TST | Slp Eff | Apneas | | Hypopneas | | AHI | RDI | Ar + Aw | | Min |
|---|---|---|---|---|---|---|---|---|---|---|---|---|
| RxI/RxE/RxO2 | Minutes | Minutes | Percent | Count | Index | Count | Index | Index | Index | Count | Index | SaO2 |
| 8/4/0 | 36 | 35.5 | 98.6 | 2 | 3.4 | 5 | 8.5 | 11.8 | 11.8 | 6 | 10.1 | 92 |
| 9/6/0 | 17 | 17 | 100 | 0 | 0 | 1 | 3.5 | 3.5 | 3.5 | 7 | 24.7 | 85 |
| 10/6/0 | 17 | 16.5 | 97.1 | 2 | 7.3 | 1 | 3.6 | 10.9 | 10.9 | 3 | 10.9 | 88 |
| 11/6/0 | 12.5 | 12.5 | 100 | 1 | 4.8 | 6 | 28.8 | 33.6 | 33.6 | 3 | 14.4 | 77 |
| 12/8/0 | 28 | 13.5 | 48.2 | 0 | 0 | 2 | 8.9 | 8.9 | 8.9 | 3 | 13.3 | 88 |
| 13/8/0 | 24 | 16 | 66.7 | 3 | 11.3 | 0 | 0 | 11.3 | 11.3 | 8 | 30 | 94 |
| 14/8/0 | 9.5 | 9.5 | 100 | 2 | 12.6 | 2 | 12.6 | 25.3 | 25.3 | 3 | 18.9 | 95 |
| 14/9/0 | 19 | 19 | 100 | 0 | 0 | 0 | 0 | 0 | 0 | 3 | 9.5 | 93 |
| 14/10/0 | 42.5 | 41.5 | 97.6 | 1 | 1.4 | 2 | 2.9 | 4.3 | 4.3 | 5 | 7.2 | 95 |
| 15/10/0 | 30.5 | 27.5 | 90.2 | 0 | 0 | 4 | 8.7 | 8.7 | 8.7 | 1 | 2.2 | 87 |
| 16/10/0 | 15.5 | 15.5 | 100 | 0 | 0 | 4 | 15.5 | 15.5 | 15.5 | 2 | 7.7 | 85 |
| 14/10/0 | 48 | 47.5 | 99. | 0 | 0 | 3 | 3.8 | 3.8 | 3.8 | 3 | 3.8 | 90 |
| 15/10/0 | 46 | 25 | 54.3 | 0 | 0 | 0 | 0 | 0 | 0 | 1 | 2.4 | 93 |
| 16/12/0 | 31.5 | 25 | 79.4 | 1 | 2.4 | 1 | 2.4 | 4.8 | 4.8 | 5 | 12 | 92 |

As a result and based upon the foregoing disclosure and representative clinical case presentations, the dual tube system of the present invention, by providing the delivery of oxygen (O₂) with the cannula 30 via the oxygen tube 34 within the shell 14 of the mask system 10 in concert with the administration of breathable gas from the machine 24 through tube 22 to the mask 10, a mask system is presented that provides a desired amount of oxygen to a patient at a low pressure. Specifically, by connecting an adjustable cannula 30 to a full face or nasal mask 30 and delivering supplemental oxygen to the nostrils rather than bleeding oxygen through the mask space or hose, the present mask system 10 provides a higher oxygen saturation during inhalation with the help of CPAP/BIPAP pressure, delivers supplemental oxygen directly to a patient's nose in order to minimize dilution and leakage to provide higher oxygenation with minimum CPAP/BIPAP pressure, and thus reduces pressure related side effects and provides higher patient compliance rates.

Furthermore, by delivering both room air/breathable gas and oxygen via a single tube assembly with oxygen tube 34 providing oxygen to the nasal cannula 10 disposed within tube 22 supplying breathable gas to the shell 14 and additionally by combining an oxygen compressor with the machine 24 which delivers breathable gas, a mask system 10 is presented which is more convenient, occupies less space, and decreases the chance of oxygen disconnection by patient movements. Finally, the disclosed mask system 10 maximizes patient comfort by delivering oxygen directly via a nasal cannula 10 through a mask 14 in order to provide better oxygenation and ultimately more effectively treat the patient with lower breathable gas pressure. Accordingly, a mask has been disclosed that, at the very least meets all the stated objectives.

What is claimed is:

1. A dual tube mask system comprising:
a frame having a shell attached to a frame, the shell having a primary port;
a machine for blowing breathable gas connected to the primary port by a tube; and
a nasal cannula that dwells within the shell and is connected to a source of oxygen.

2. The system of claim 1 wherein the cannula is connected to the source of oxygen by an oxygen tube that extends from the cannula through the primary port and into the tube connecting the machine to the primary port.

3. The system of claim 1 wherein the shell has a secondary port.

4. The system of claim 3 wherein the cannula is connected to an inner end of the secondary port and an oxygen tube is connected to an outer end of the secondary port.

5. The system of claim 3 wherein the cannula extends through the secondary port and is connected to an oxygen tube.

6. The system of claim 3 wherein the cannula is connected to an oxygen tube and the oxygen tube extends through the secondary port.
